(19) United States
(12) Patent Application Publication
(10) Pub. No.: US 2015/0099942 A1
(43) Pub. Date: Apr. 9, 2015

(54) VASCULAR SECUREMENT CATHETER WITH IMAGING

(71) Applicant: VOLCANO CORPORATION, San Diego, CA (US)

(72) Inventor: Philippe K. Edouard, San Diego, CA (US)

(21) Appl. No.: 14/497,677

(22) Filed: Sep. 26, 2014

Related U.S. Application Data
(60) Provisional application No. 61/887,207, filed on Oct. 4, 2013.

(51) Int. Cl. A61B 17/00; A61B 5/00; A61B 1/018; A61B 8/00; A61N 7/02; A61B 1/04; A61M 25/01; A61B 5/026; A61B 5/145; A61B 5/0205; A61F 2/82; A61B 17/064; A61N 1/04; A61F 2/01; A61F 2/24; A61F 2/07; A61B 17/08; A61B 17/86; A61B 8/12

(52) U.S. Cl. CPC A61B 17/00234; A61B 8/12; A61B 5/0066; A61B 5/0075; A61B 5/0084; A61B 1/018; A61B 8/445; A61N 7/022; A61B 8/4494; A61B 1/04; A61M 25/0108; A61B 5/026; A61B 5/14542; A61B 5/02055; A61F 2/82; A61B 17/064; A61N 1/04; A61F 2/01; A61F 2/24; A61F 2/07; A61B 17/083; A61B 17/86; A61N 2007/0052
USPC 600/301; 600/467; 600/427; 600/473; 600/476; 600/104; 600/439; 600/424; 600/505; 600/486; 600/364; 600/549; 600/407

(57) ABSTRACT

A catheter for delivering securing elements to a tissue, such as a blood vessel, and imaging the tissue before, after, or during the imaging. The catheters incorporate intravascular ultrasound (IVUS) imaging, including Focused Acoustic Computed Tomography (FACT), as well as optical coherence tomography (OCT).

### RELATED APPLICATIONS

This application claims priority to U.S. Provisional No. 61/887,207, filed Oct. 4, 2013, which is incorporated herein by reference in its entirety.

### FIELD OF THE INVENTION

The invention relates to catheters configured to accomplish multiple tasks at a treatment site, such as imaging, diagnostic measurement, and device delivery.

### BACKGROUND

Aneurysms of the aorta primarily occur in abdominal region, usually in the infrarenal area between the renal arteries and the aortic bifurcation. Aneurysms can also occur in the thoracic region between the aortic arch and renal arteries. The rupture of an aortic aneurysm results in massive hemorrhaging and has a high rate of mortality. Open surgical replacement of a diseased or damaged section of vessel can eliminate the risk of vessel rupture, however there is a non-negligent mortality rate associated with this open surgery itself, and the recovery times are substantial. During open surgical repair, the diseased or damaged section of vessel is removed and a prosthetic graft, made either in a straight of bifurcated configuration, is installed and then permanently attached and sealed to the ends of the native vessel by suture. The prosthetic grafts for these procedures are usually unsupported woven tubes and are typically made from polyester, ePTFE or other suitable materials. The grafts are longitudinally unsupported so they can accommodate changes in the morphology of the aneurysm and native vessel.

Endovascular aneurysm repair has been introduced to overcome the problems associated with open surgical repair Like many corrective endovascular procedures, however, aortic aneurism repairs typically require multiple passes and a variety of different catheters to evaluate, repair, and then re-evaluate the repair. Typically, the aneurysm is bridged with an intraluminally-delivered vascular prosthesis. Then the prosthetic graft is delivered in a collapsed state on a catheter through the femoral artery. These grafts are often designed with a fabric material attached to a metallic scaffolding (stent) structure, which expands to the internal diameter of the vessel. The grafts may additionally include barbs or hooks to secure the graft to the tissue.

With current intraluminal aneurysm repair, each step requires a separate specialized catheter. For example, a patient having an aneurysm will have a guidewire placed into the aorta and then an imaging catheter will be delivered to the aorta to evaluate the site. After evaluation, the site may be re-imaged with angiography to verify the location of the defect. The imaging catheter will then be removed, and a new graft delivery catheter will be delivered on the original guidewire. Once delivered, the prosthetic graft can be deployed at the site of the aneurysm. The graft delivery catheter is then removed, and the imaging catheter is replaced to evaluate the success of the prosthesis.

In most instances, intraluminally-deployed grafts are not sutured to the native vessel, but rather rely on barbs extending from the stent, or the radial expansion force of the stent to hold the graft in position. Compared to suture, however, barbs do not provide the same level of attachment, and they can damage the native vessel upon deployment. Furthermore, it is difficult to control the location of each barb in the graft as it deploys, and it is possible for a barb to travel through the vessel wall and damage an adjoining organ. For these reasons, it is often necessary to perform additional imaging after barbed graft delivery to evaluate the condition of the vessel and its surrounds.

### SUMMARY

The invention facilitates advanced aneurysm treatments by providing catheters that allow vascular imaging and placement of fasteners with a single catheter. The combination of functionality makes it easier for a provider to evaluate the site of the fastener placement prior to anchoring the fastener to the vessel. The invention additionally makes it possibly to instantly evaluate the positioning of the fastener and the health or thickness of the tissue to which the fastener is affixed. Because the procedures require no, or fewer, catheter exchanges, the procedure can be completed faster, thereby reducing a patient's exposure to contrast and x-rays, while reducing the cumulative risk of perforation. The imaging may be intravascular ultrasound (IVUS), including focused acoustic computed tomography (FACT), optical coherence tomography (OCT), or visible imaging.

The invention is not limited to cardiovascular applications, however, because catheters according to the invention generally provide an ability to image tissue(s), deliver a fastener, and evaluate the success of the delivery. For example, using a device of the invention, it is possible to obstruct a body lumen such as the vas deferens. Additionally, because the devices of the invention have such a small diameter, the site can be reached through an entry such as the urethra. In an embodiment, the device is capable of imaging the tissue with intravascular ultrasound (IVUS). In another instance, the invention is an implantable device capable of imaging a tissue with optical coherence tomography.

These and other aspects, advantages, and features of the invention will be better understood with reference to the following drawings and description.

### BRIEF DESCRIPTION OF THE DRAWINGS

FIG. 1A depicts a side view of the distal end of a catheter of the invention;

FIG. 1B depicts a top view of the distal end of a catheter of the invention;

FIG. 2A depicts a side view of the distal end of a catheter of the invention;

FIG. 2B depicts a top view of the distal end of a catheter of the invention;

FIG. 3A depicts a side view of the distal end of a catheter of the invention;

FIG. 3B depicts a top view of the distal end of a catheter of the invention;

FIG. 4A depicts a side view of the distal end of a catheter of the invention;

FIG. 4B depicts a top view of the distal end of a catheter of the invention;

FIG. 5 depicts a concave micromachined piezoelectric ultrasound element adapted for focused acoustic computed tomography (FACT);

FIG. 6 depicts a system including a catheter of the invention;

FIG. 7A is a diagram of components of an optical coherence tomography (OCT) subsystem;

FIG. 7B is a diagram of the imaging engine shown in FIG. 7A; and

FIG. 8 is a diagram of a light path in an OCT system of certain embodiments of the invention.

### DETAILED DESCRIPTION

The invention provides advanced intraluminal catheters capable of imaging tissues and delivering fasteners to the tissues with a single device. In some embodiments, the catheters additionally allow monitoring the environment in proximity to the tissues receiving the fasteners. The catheters of the invention may use “conventional” IVUS components, such as piezoelectric transducers, or the devices may use advanced IVUS components, such as piezoelectric micromachined ultrasonic transducers (PMUTs), or capacitive micromachined ultrasonic transducers (CMUTs). In some embodiments, the catheters may use optical coherence tomography (OCT). The catheters lend themselves to methods for the treatment of tissues in need thereof as well as systems including the devices of the invention.

Using the catheters of the invention, a variety of target tissues can be imaged, diagnosed, treated, and evaluated with the devices, methods, and systems of the invention. In particular the invention is useful for treating tissues that are accessible via the various lumens of the body, including, but not limited to, blood vessels, vasculature of the lymphatic and nervous systems, structures of the gastrointestinal tract (lumens of the small intestine, large intestine, stomach, esophagus, colon, pancreatic duct, bile duct, hepatic duct), lumens of the reproductive tract (vas deferens, uterus and fallopian tubes), structures of the urinary tract (urinary collecting ducts, renal tubules, ureter, and bladder), and structures of the head and neck and pulmonary system (sinuses, parotid, trachea, bronchi, and lungs). Accordingly, the devices, methods, and systems of the invention may be beneficial in the treatment of a number of disorders, including, but not limited to, atherosclerosis, ischemia, coronary blockages, thrombi, occlusions, stenosis, and aneurysms. The devices, methods, and systems can also be used to treat cancer, inflammatory disease (e.g., autoimmune disease, arthritis), pain, and genetic disorders.

As described in the background, there is a need for techniques for delivering and anchoring a prosthetic graft, e.g., an endograft. In an embodiment, the graft can include a support frame or scaffold. Suitable grafts are available from manufacturers such as Cook Medical Devices and Medtronic. The scaffold may be elastic, e.g., comprised of a shape memory alloy elastic stainless steel, or the like. For elastic scaffolds, expanding typically comprises releasing the scaffolding from a constraint to permit the scaffold to self-expand at the implantation site. Alternatively, placement of a tubular catheter, delivery sheath, or the like over the scaffold can serve to maintain the scaffold in a radially reduced configuration. In this arrangement, self-expansion of the scaffold is achieved by pulling back on the radial constraining member, to permit the scaffold to assume its larger diameter configuration. In alternative embodiments, the scaffold may be constrained in an axially elongated configuration, e.g., by attaching either end of the scaffold to an internal tube, rod, catheter or the like. This maintains the scaffold in the elongated, reduced diameter configuration. The scaffold may then be released from such axial constraint in order to permit self-expansion.

In some embodiments, the scaffold may be formed from a malleable material, such as malleable stainless steel of other metals. Expansion may then comprise applying a radially expansive force within the scaffold to cause expansion, e.g., inflating a scaffold delivery catheter within the scaffold in order to affect the expansion. In this arrangement, the positioning and deployment of the endograft can be accomplished by the use of an expansion means either separate or incorporated into the deployment catheter. This will allow the endograft to be positioned within the vessel and partially deployed while checking relative position within the vessel. The expansion can be accomplished either via a balloon or mechanical expansion device. Additionally, this expansion stabilizes the position of the endograft within the artery by resisting the force of blood on the endograft until the endograft can be fully deployed.

The graft may have a wide variety of conventional configurations. It may comprise a fabric or some other blood semi-impermeable flexible barrier which is supported by the scaffold, which can take the form of a stent structure. The stent structure can have any conventional stent configuration, such as zigzag, serpentine, expanding diamond, or combinations thereof. The stent structure may extend the entire length of the graft, and in some instances can be longer than the fabric components of the graft. Alternatively, the stent structure can cover only a small portion of the prosthesis, e.g., being present at the ends. The stent structure may have three or more ends when it is configured to treat bifurcated vascular regions, such as the treatment of abdominal aortic aneurysms, when the stent graft extends into the iliac arteries. In certain instances, the stent structures can be spaced apart along the entire length, or at least a major portion of the entire length, of the stent-graft, where individual stent structures are not connected to each other directly, but rather connected to the fabric or other flexible component of the graft.

The graft deployment typically involves a prosthesis delivery catheter used to deliver the graft in a collapsed configuration. Once released, the graft typically self-deploys, with stabilization struts holding the self-expanding stent structure in position against the vessel wall. The struts support the stent structure (and thus the overall prosthesis) against the force of blood flow through the vessel during prosthesis deployment. The devices, methods, and systems of the invention can also be used to administer therapy with a catheter. The devices can be used for angioplasty, such as balloon angioplasty. The devices can be used for ablation, such as balloon ablation, or probe ablation. The devices can be used to aspirate or remove tissues. The devices can be used for medical device placement, such as stents, struts, valves, filters, pacemakers, or radiomarkers. The devices, methods, and systems of the invention may be used to administer more than one therapy of combinations of therapies and therapeutics, e.g., drugs. For example, a solution delivered to a tissue in need of treatment may comprise a thrombolytic drug and aspiration.

As discussed previously, the devices of the invention can be used to secure prosthetic grafts against movement within the vasculature. The fasteners are typically deployed after the prosthesis has been initially placed. For example, initial placement of the prosthesis may be achieved by self-expansion or balloon expansion, after which the prosthesis is secured or anchored in place by the introduction of a plurality of individual fasteners. In some instances, the fasteners will be placed only through the fabric of the prosthesis, i.e., avoiding the scaffold structure. Alternately, the fasteners may be introduced into and through portions of the scaffold structure itself. The prosthesis may include preformed receptacles, apertures, or grommets, which are specially configured to receive the fasteners. In other embodiments, the fasteners may be introduced both through the fabric and through the scaffold structure. The fasteners can be introduced singly, i.e., one at a time, in a circumferentially spaced-apart pattern over an interior wall of the prosthesis.

A variety of fasteners may be delivered with catheters of the invention. In one embodiment, the fasteners are helical fasteners that can be rotated and “screwed into” a graft and a vessel wall, or simply used to reinforce weakened tissues. A desired configuration for the helical fastener is an open coil, much like a coil spring. This configuration allows the fastener to capture a large area of tissue, which results in significantly greater holding force than conventional staples, without applying tissue compression, which can lead to tissue necrosis. In an embodiment, the proximal end of the helical fastener includes an L-shaped leg of the coil bisecting the fastener diameter. The leg of the coil comes completely across the diameter to prevent the fastener from being an open coil and to control the depth of penetration into the tissue. In addition, the leg of the coil can be attached to a previous coil to strengthen the entire structure and provide a more stable drive attachment point for the fastener applier. This attachment could be achieved via welding, adhesive or any other suitable means.

Other fasteners, such as screws, ties, clips, or staples can be delivered with a device of the invention. The fasteners can be made from stainless steel or other types of implantable metal, however it is also envisioned that the fasteners in the above descriptions could be made from implantable polymers, or from a biodegradable polymers, or combinations of all materials above.

In practice, intravascular catheters are delivered to a tissue of interest via an introducer sheath placed in the radial, brachial or femoral artery. The introducer is inserted into the artery with a large needle, and after the needle is removed, the introducer provides access for guidewires, catheters, and other endovascular tools. An experienced cardiologist can perform a variety of procedures through the introducer by inserting tools such as balloon catheters, stents, or cauterization instruments. When the procedure is complete, the introducer is removed, and the wound can be secured with suture tape. Catheter lengths vary up to 400 cm, depending on the anatomy and work flow. Catheters of the invention are typically greater than 50 cm in length, e.g., greater than 100 cm in length, e.g., greater than 150 cm in length, e.g., greater than 200 cm in length, e.g., greater than 250 cm in length, e.g., greater than 300 cm in length. The ends of the catheter are denoted as distal (far from the user, i.e., inside the body) and proximal (near the user, i.e., outside the body).

Importantly, the catheters of the invention are able to image a tissue, i.e., cardiovascular tissue, prior to treatment. In particular, the invention provides devices, systems and methods for imaging tissue using intravascular ultrasound (IVUS). IVUS uses a catheter with an ultrasound transducer disposed at the distal end. Systems for IVUS are also discussed in U.S. Pat. No. 5,771,895, U.S. Pat. Pub. 2009/0284332, U.S. Pat. Pub. 2009/0195514 A1, U.S. Pat. Pub. 2007/0232933, and U.S. Pat. Pub. 2005/0249391, the entire contents of each of which are incorporated herein by reference. In advanced embodiments, the IVUS systems of the invention incorporate focused acoustic computed tomography (FACT), which is described in WO2014/109879, incorporated herein by reference in its entirety. In FACT embodiments, the ultrasonic energy used to image the tissue is focused to achieve deeper penetration into tissues, and higher contrast between different types of tissue. In some embodiments, multiple ultrasound bandwidths are used to improve resolution of structure and composition.

In some embodiments, the devices are capable of imaging tissues with optical coherence tomography (OCT), which uses interferometric measurements to determine radial distances and tissue compositions. Systems for OCT imaging are discussed in U.S. Pat. No. 7,813,609 and US Patent Publication No. 20090043191, both of which are incorporated herein by reference in their entireties.

The disclosed catheters are commonly used in conjunction with guidewires. Guidewires are known medical devices used in the vasculature or other anatomical passageway and act as a guide for other devices, e.g., a catheter. Typically, the guidewire is inserted into an artery or vein and guided through the vasculature under fluoroscopy (real time x-ray imaging) to the location of interest. (As discussed previously, some procedures require one or more catheters to be delivered over the guidewire to diagnose, image, or treat the condition.) Guidewires typically have diameters of 0.010″ to 0.035″, with 0.014″ being the most common. Guidewires (and other intravascular objects) are also sized in units of French, each French being ⅓ of a mm or 0.013″. Guidewire lengths vary up to 400 cm, depending on the anatomy and work flow. Often a guidewire has a flexible distal tip portion about 3 cm long and a slightly less flexible portion about 30 to 50 cm long leading up to the tip with the remainder of the guidewire being stiffer to assist in maneuvering the guidewire through tortuous vasculature, etc. The tip of a guidewire typically has a stop or a hook to prevent a guided device, e.g., a catheter from passing beyond the distal tip. In some embodiments, the tip can be deformed by a user to produce a desired shape.

Advanced guidewire designs include sensors that measure flow and pressure, among other things. For example, the FLOWIRE® Doppler GuideWire, available from Volcano Corp. (San Diego, Calif.), has a tip-mounted ultrasound transducer and can be used in all blood vessels, including both coronary and peripheral vessels, to measure blood flow velocities during diagnostic angiography and/or interventional procedures. Advanced guidewires, such as FLOWIRE® can be used with the described inventions. In some instances, an advanced guidewire can be used to supplement the capabilities of the devices of the invention. In some instances, an advanced guidewire can be used to replace a capability (e.g., flow sensing) of a disclosed device. In some instances, and advanced guidewire is incorporated into a system of the invention, e.g., additionally including a catheter described below.

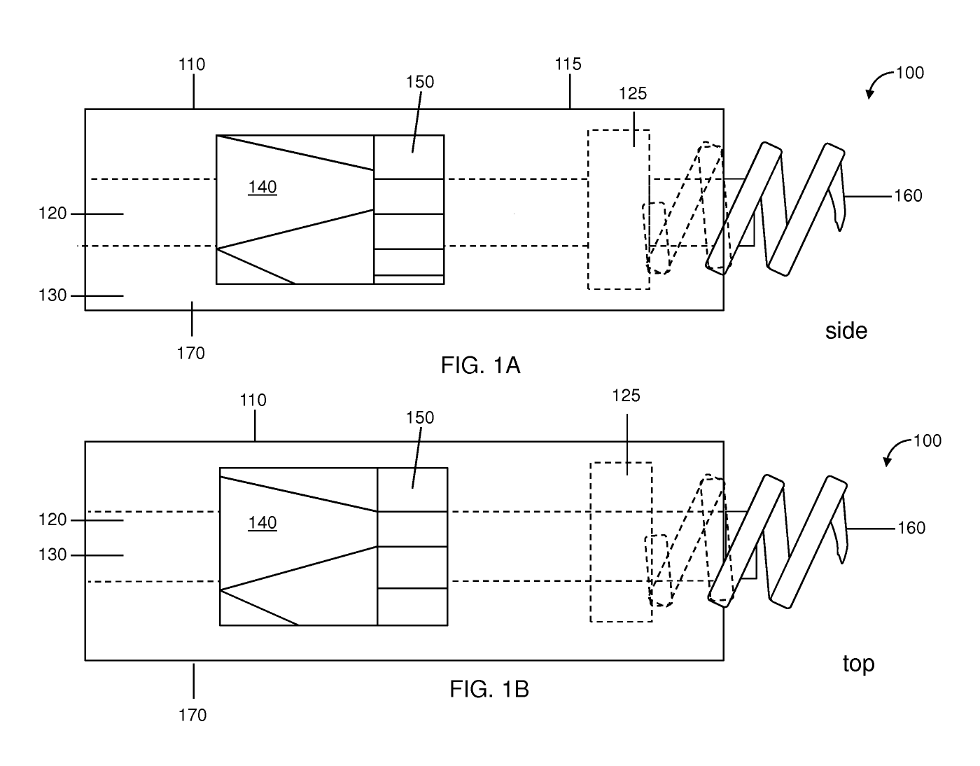

Importantly, catheters of the invention can be used to deliver fasteners 160 located at the distal end of the catheter, as shown in FIGS. 1-4. The drive mechanism 120 includes a driver head 125 that is coupled to the drive mechanism 120. The drive mechanism 120 is typically coupled to a motor and a controller at the proximal end of the device. The coupling between the driver head 125 and fastener 160 can take different forms—e.g., magnets, graspers, or other suitable mechanical connection.

The distal end 110 of a catheter of the invention is shown in FIGS. 1A and 1B. FIG. 1A shows a side view of an imaging/delivery catheter 100 that uses piezoelectric elements as ultrasound transducers 140 and ultrasound receivers 150 to produce and receive ultrasound energy for imaging. Catheter 100 includes a proximal end (not shown), a mid-body (not shown), and a distal end 110 including a distal tip 115. The ultrasound transducers 140 are constructed from piezoelectric components that produce sound energy at 20-50 MHz. The ultrasound transducers 140 are known in the field of intravascular ultrasound imaging, and are commercially available from suppliers such as Blatek, Inc. (State College, Pa.).

As shown in FIGS. 1A and 1B, the ultrasound transducers 140 are configured in a phased array, that is, each ultrasound receiver 150 is a separate piezoelectric element that produces ultrasound energy. Similarly, each ultrasound receiver 150 is an independent element configured to receive ultrasound energy reflected from the tissues to be imaged. Alternative embodiments of the ultrasound transducers 140 and the ultrasound receivers 150 may use the same piezoelectric components to produce and receive the ultrasonic energy, for example, by using pulsed ultrasound. Another alternative embodiment may incorporate ultrasound absorbing materials and/or ultrasound lenses to increase signal to noise. Both the ultrasound transducers 140 and the ultrasound receivers 150 have electrical connectors (not shown) that extend from the transducers 140 and receivers 150 to the proximal end of the device to provide power, and to provide and receive ultrasound signals. As can be seen more clearly in FIG. 1B, the transducers 140 and receivers 150 are coaxially located with the drive mechanism 120 to maximize the clearance for the drive mechanism 120 with respect to the diameter of the distal end 110 of the device.

Other sensors, such as temperature and pressure sensors can also be accommodated in distal end 110. For example, the distal end 110 may include a thermocouple, a thermistor, or a temperature diode to measure the temperature of the surroundings. The distal end 110 may include a pressure sensor, such as a piezoelectric pressure sensor, or a semiconductor pressure sensor. The distal end 110 may also include one or more elements to perform spectroscopic measurements, e.g., infrared absorption spectroscopy, visible wavelength absorption spectroscopy, fluorescence spectroscopy, or Raman spectroscopy. In some embodiments, the spectroscopic measurement will rely on collecting back-scattered or fluorescent light. In some embodiments, the spectroscopic measurements can be made with optical elements that are also used to make OCT measurements. In some embodiments, the distal end 110 of the catheter will include an optical pathway that is in fluid communication with the surroundings of the catheter, thereby allowing direct absorption measurements, for example, visible absorbance spectroscopy.

Using spectroscopic methods, it is possible to probe a tissue, or the environment around the tissue, for the presence of specific chemical species indicative of the health of the tissue (or the surroundings) or indicative of the efficacy of an administered treatment. The chemical species may include, for example, calcium ions or sodium ions. The methods may also be used to monitor oxygen content of the blood or to determine a level of hemoglobin, for example. In some instances, a dye, i.e., an intercalating dye, can be used in conjunction with the spectroscopic methods to determine the presence of free nucleic acids.

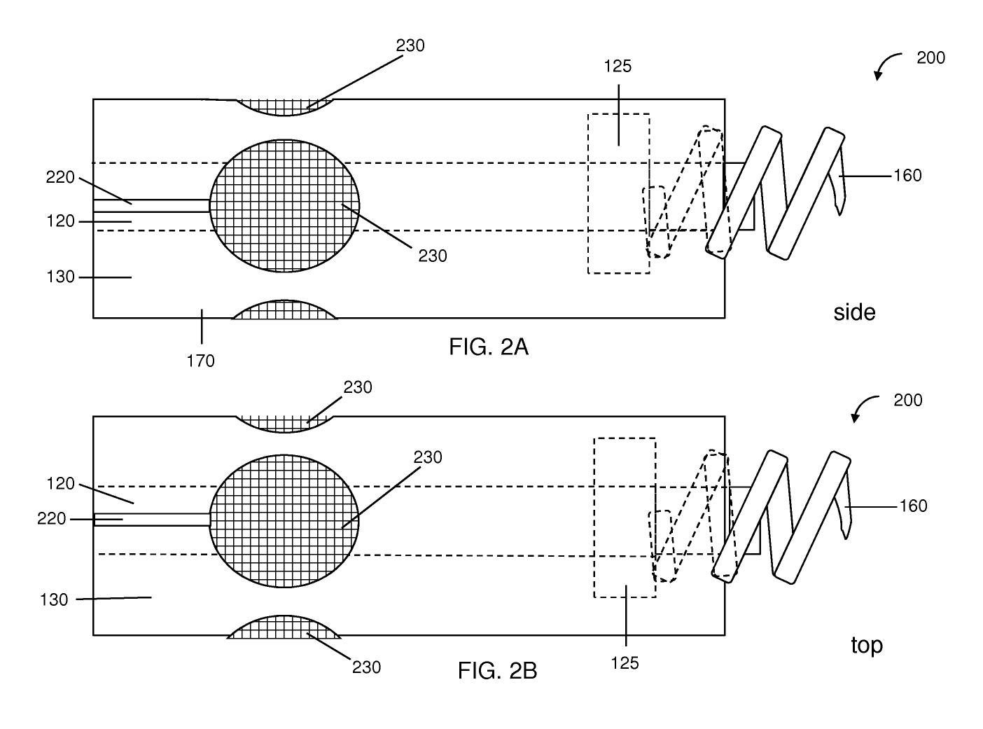

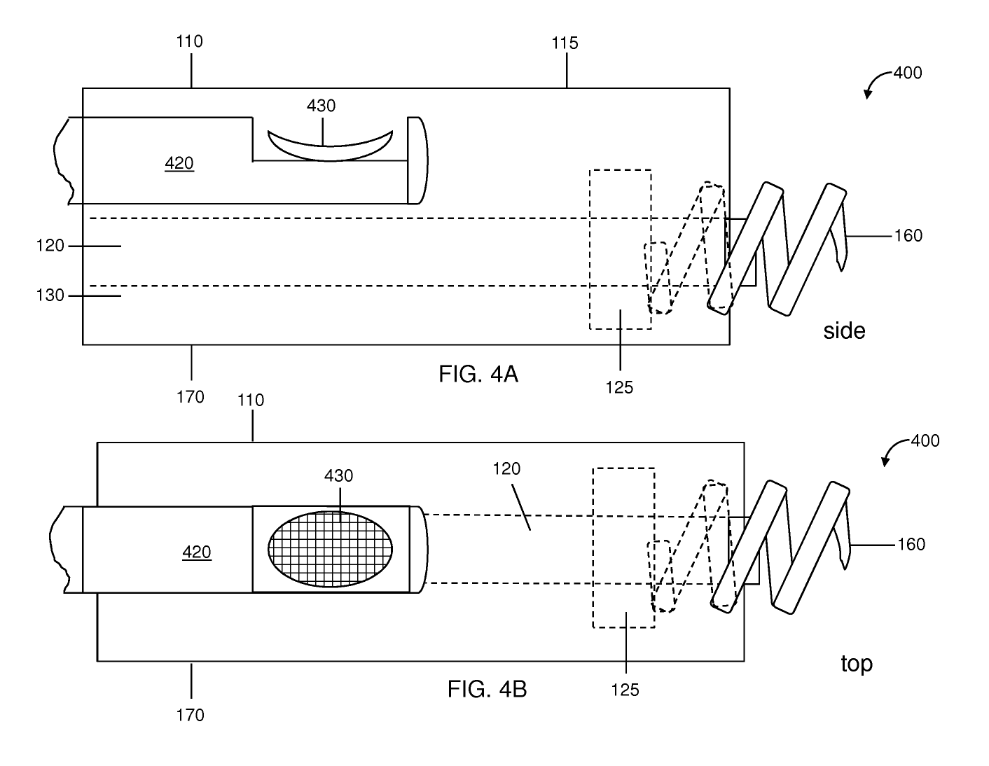

A different embodiment of the imaging/delivery catheter 200 is shown in FIGS. 2A and 2B. FIG. 2A shows a side view of an imaging/delivery/evaluation catheter 200 that uses a plurality of piezoelectric micromachined ultrasound transducers (PMUTs) 230 for imaging. The catheter 200 of FIGS. 2A and 2B includes a proximal end (not shown), a mid-body (not shown), and a distal end 110 including a distal tip 115. The distal end 110 includes drive mechanism 120 connected to driver head 125 connected to fastener 160. The PMUTs 230 shown in FIGS. 2A and 2B comprise fixed arrays of ultrasound transducers. However, in other embodiments, i.e., as shown in FIGS. 4A and 4B, the PMUT may be coupled to a rotational stage that is configured for pull-back imaging. An exemplary PMUT 230 used in IVUS catheters may include a polymer piezoelectric membrane, such as that disclosed in U.S. Pat. No. 6,641,540, incorporated herein by reference in its entirety. The PMUT 230 may provide greater than about 70% bandwidth, i.e., greater than about 75% bandwidth between from about 10 MHz to about 50 MHz of ultrasound for optimum resolution. In some embodiments, the PMUT 230 will be accompanied by a spherically aperture (not shown) for focusing. The PMUT package may also include a housing 220 having the PMUT 230 and associated circuitry disposed therein, such as an application-specific integrated circuit (ASIC). In yet other embodiments, transducer assembly may include a capacitive micro-machined ultrasonic transducer (“CMUT”) (not shown).

While not shown in the figures, the described catheters may include radiopaque markers at various locations on or within the catheter to identify structures, e.g., with fluoroscopy. The radiopaque markers will be small in most instances, having a longitudinal dimension of less than 5 mm, e.g., less than 4 mm, e.g., less than 3 mm, e.g., less than 2 mm, e.g., less than 1 mm. The radiopaque markers will be at least 0.2 mm, e.g., at least 0.3 mm, e.g., at least 0.4 mm, e.g., at least 0.5 mm. The radiopaque markers may vary in axial size or diameter, depending upon their shape; however markers necessarily will be small enough to fit within a catheter, e.g., catheter 100, 200, 300, or 400. The radiopaque markers may be constructed from any material that does not transmit x-rays and has suitable mechanical properties, including platinum, palladium, rhenium, tungsten, and tantalum.

Other catheter embodiments may combine delivery therapies with optical coherence tomography (OCT) imaging. In OCT, light from a broad band light source or tunable laser source is split by an optical fiber splitter with one fiber directing light to the distal end of a catheter, e.g., for imaging a tissue, and the other fiber directing light to a reference mirror. The distal end of the optical fiber is interfaced with the distal end of a catheter for interrogation of tissues, etc. The light emerges from the optical fiber and is reflected from the tissue being imaged. The reflected light from the tissue is collected with the optical fiber and recombined with the signal from the reference mirror forming interference fringes (measured by a detector) allowing precise depth-resolved imaging of the tissue on a micron scale.

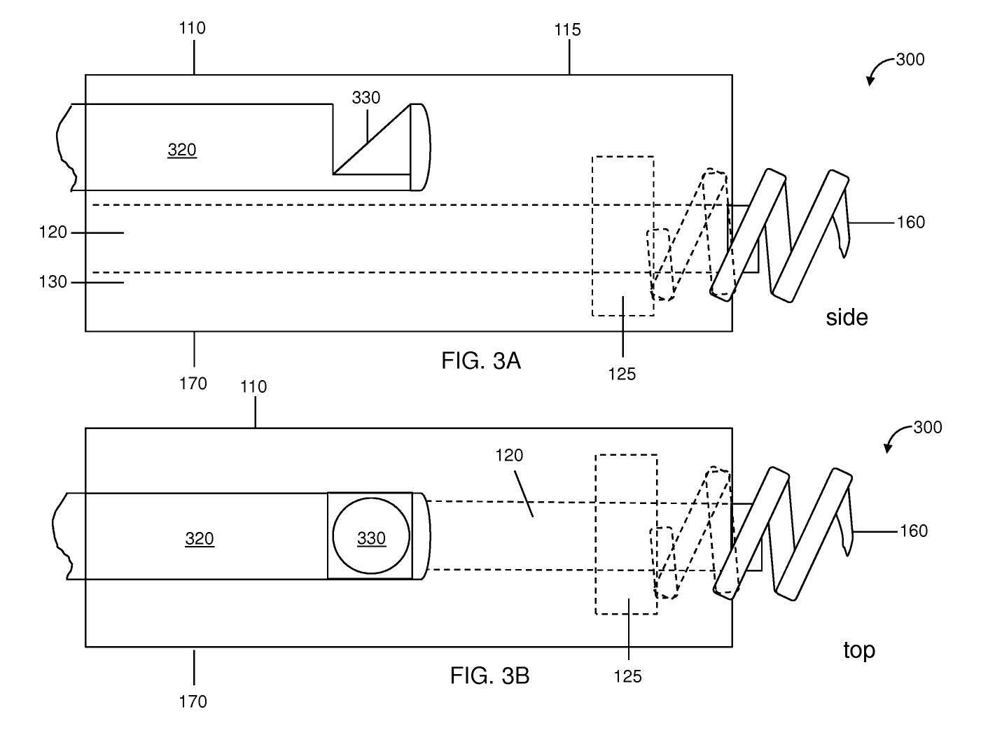

An alternative embodiment, configured to imaging tissues with OCT is shown in FIGS. 3A and 3B. FIG. 3A shows a side view of an imaging/delivery/evaluation catheter 300 adapted for rotational OCT imaging, allowing a user to evaluate tissues before and after treatment. Catheter 300 includes a proximal end (not shown), a mid-body (not shown), and a distal end 110 including a distal tip 115. The distal end 110 includes driving mechanism 120 connected to a driver head 125, coupled to fastener 160.

Catheter 300 includes rotational element 320 and mirror 330 which direct light out of an optical fiber (not shown) and collect light that scatters off of the imaged tissue for the purpose of creating tissue measurements using the technique of optical coherence tomography (OCT). OCT typically uses a super diode source or tunable laser source emitting a 400-2000 nm wavelength, with a 50-250 nm bandwidth (distribution of wave length) to make in-situ tomographic images with axial resolution of 2-20 μm and tissue penetration of 2-3 mm. The near infrared light sources used in OCT instrumentation can penetrate into heavily calcified tissue regions characteristic of advanced coronary artery disease. With cellular resolution, application of OCT may be used to identify other details of the vulnerable plaque such as infiltration of monocytes and macrophages. In short, application of OCT can provide detailed images of a pathologic specimen without cutting or disturbing the tissue.

The rotational element 320 may only rotate, or the rotational element 320 may translate and rotate, i.e., incorporating pull-back imaging. The principles of pull-back OCT devices are described in detail in U.S. Pat. No. 7,813,609 and US Patent Publication No. 20090043191, both of which are incorporated herein by reference in their entireties.

Another alternative embodiment, capable of imaging the tissues with focused acoustic computed tomography (FACT) is shown in FIGS. 4A and 4B. FIG. 4A shows a side view of an imaging/delivery/evaluation catheter 400 that is configured to use pull-back FACT to evaluate tissues before and after treatment. Catheter 400 includes a proximal end (not shown), amid-body (not shown), and a distal end 110 including a distal tip 115. The distal end 110 includes driving mechanism 120 connected to a driver head 125, coupled to fastener 160.

Catheter 400 includes rotational element 420 and concave ultrasound transducer 430 that directs a focused beam of ultrasonic energy from the catheter and collects ultrasonic echoes that are returned from the tissue. The ultrasound echoes are then used to construct images of the tissues with improved contrast and depth features, i.e., using the FACT techniques described above. The rotational element 420 may only rotate, or the rotational element 420 may translate and rotate, i.e., incorporating pull-back imaging. The rotational element 420 may additionally include power, control, and signal processing circuitry for the transducer 430, The principles of pull-back imaging catheters are described in detail in U.S. Pat. No. 7,813,609 and US Patent Publication No. 20090043191, both of which are incorporated herein by reference in their entireties.

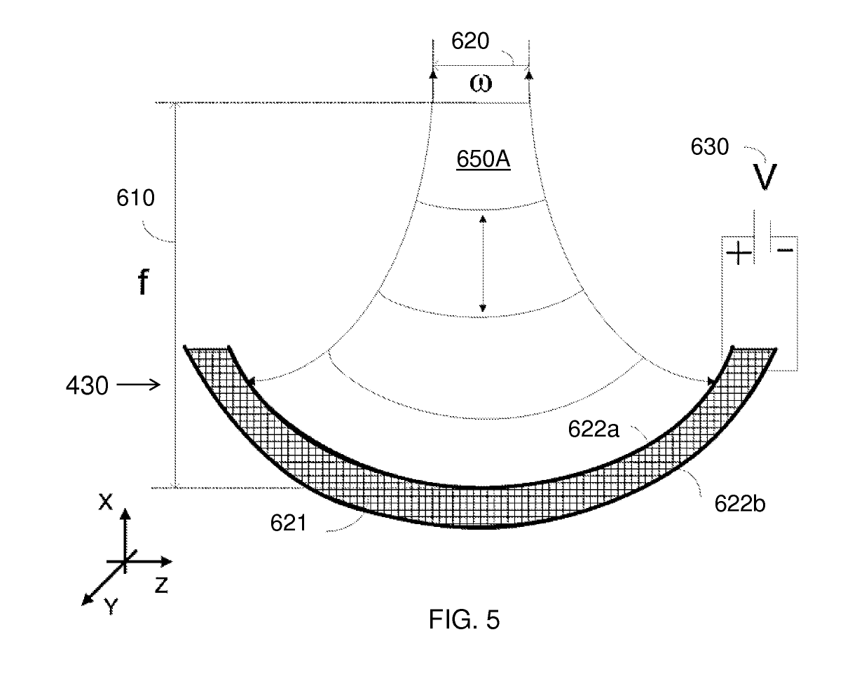

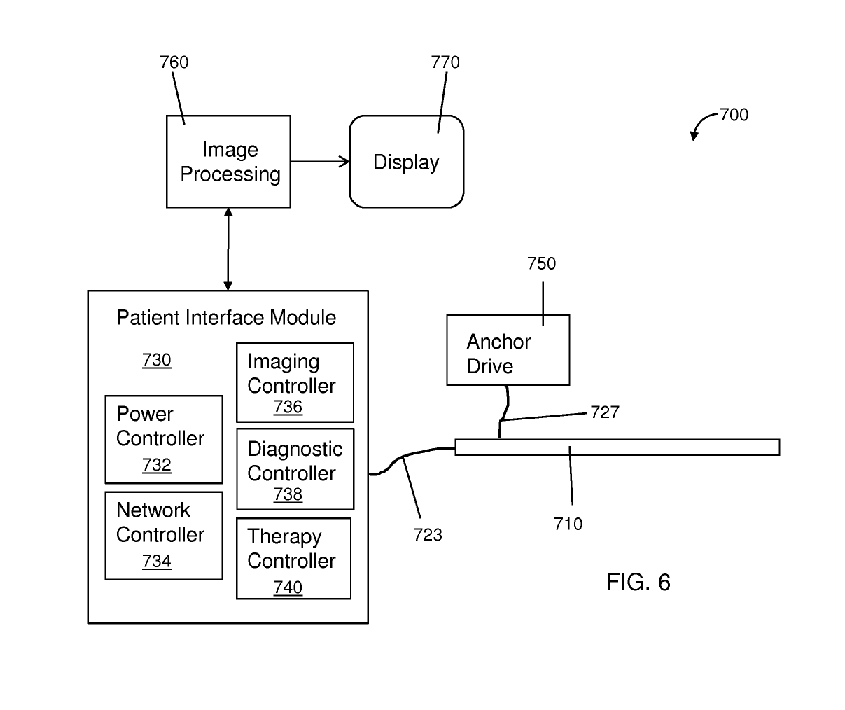

The function of concave ultrasound transducer 430 of catheter 400 is shown in greater detail in FIG. 5. Transducer 430 includes a polymeric layer 621 having a first adjacent conductive layer 622a and a second adjacent conductive layer 622b. Polymeric layer 621 includes a piezoelectric polymer material made into a concave shape as depicted in FIG. 6. In some embodiments, the polymer used in polymeric layer 621 may be a ferroelectric polymer such as polyvinylidene fluoride (PVDF). Further according to some embodiments, polymeric layer 621 may include PVDF-co-trifluoroethylene (PVDF-TrFE) as a piezo-electric material. A voltage 630 (V) is applied between conductive layers 622a and 622b in order to generate a focused ultrasound beam 650A. Likewise, incident ultrasonic energy may impinge on polymeric layer 621 and produce a surface change leading to a voltage difference V 630 between conductive layers 622a and 622b. In some embodiments, the concavity of transducer 430 may be a section of a sphere. In some embodiments, the concavity of transducer 430 is directed radially outward, in a plane perpendicular to the catheter 400. The structure of the transducer assembly including backing, electrodes, and matching layers may determine the acoustic frequency bandwidth of transducer 430. The viscoelastic properties of the polymer material may also determine the acoustic frequency bandwidth of transducer 430. In some embodiments, the transducer 430 will be capable of producing an ultrasonic signal at a frequency between 5 and 135 MHz. In some instances, the transducer 430 will produce a broad bandwidth of ultrasonic frequencies. In other instances, the transducer 430 will produce a narrow bandwidth of ultrasonic frequencies, e.g., with a FWHM of 20 MHz, centered at 50 MHz. In other instances, the transducer 430 will produce a variety of narrow bandwidths to achieve better contrast between materials with different compositions, i.e., between calcified and non-calcified vascular tissue.

In rotational IVUS embodiments, transducer 430 rotates along with the rotational element 420, thus sweeping focused beam 650A radially in the XY plane, as shown in FIG. 5. In alternative embodiments, transducer 430 may include a planar polymeric layer 621, and an acoustic lens (not shown) may be placed adjacent to the now-planar transducer 430. Accordingly, focused acoustic beam 650A may be generated by acoustic wave refraction through the lens. Alternatively, or additionally, the material forming the catheter may have an engineered acoustic impedance, thereby focusing the acoustic wave propagating through the round wall of the catheter.

In some instances the focal distance 610 is determined from the curvature of the surface formed by transducers 430 and the refractive index of the propagation medium of focused acoustic beam 650A. Typically, the propagation medium is blood, plasma, a saline solution, or some other bodily fluid. In some embodiments, focal distance may be as long as 10 nm, or more. Thus, the tissue penetration depth of focused ultrasonic beams 650A may be 5 mm, 10 mm, or more. Focal distance 610 and focal waist 620 may also be determined by the curvature of the aperture. In some embodiments focused acoustic beam 650A, may include a plurality of acoustic frequencies in a frequency bandwidth. The frequency bandwidth may be determined by the polymer material and the shape of polymeric layer 221. Further according to some embodiments, the material and shape of distal portion 115 of sheath 110 may be selected to match the acoustic impedance of the materials in transducer 430 and the target structure (e.g., blood vessel wall). Impedance matching of the acoustic signal across all elements in the distal portion of catheter 400 is desirable to enhance the response of transducer 430 to the acoustic echo coming from the blood vessel wall.

As an alternate to the catheter 400 shown in FIGS. 4A and 4B, a concave transducer may be used in conjunction with a rotating mirror similar to the rotating mirror 330 shown in FIGS. 3A and 3B. Accordingly, the output of the transducer or a reflecting element may be oriented to generally align with the longitudinal axis of the catheter, and the mirror may be swept through an arc to generate annular images transverse to the catheter.

In other embodiments, visible imaging may be used in combination with a catheter-delivered fastener system. Visible imaging may be used in addition to, or in lieu of, the imaging modalities discussed above, e.g., IVUS, FACT, and OCT. Visible imaging typically allows a user to “see” the tissue in the visible wavelengths using visible image collection devices, such as a camera, optical fibers coupled to a lens, or CCD arrays. A catheter of the invention, using visible imaging, may additionally comprise an illumination source, e.g., a light, to illuminate the tissue so that it can be visualized. The visible imaging may also be coupled to image processing and recording equipment so that the visible images can be analyzed and stored. In some embodiments, the images will be processed in real-time and output to a display to allow a user to identify anatomical features during a procedure.

A system 700, including a multifunction catheter 710, is shown in FIG. 6. As discussed above, the catheter 710 may include a pigtail 723, including the needed electrical/optical connections, and a fluid delivery branch 727. The pigtail 723 is connected to a Patient Interface Module (PIM) 730 that provides the needed signals to produce acoustic energy for imaging and therapy, and receives the return signals to produce images of the tissues or to diagnose the anatomical environment in proximity to the tissues.

As shown in FIG. 6, the PIM 730 comprises multiple components, each controlling an aspect of the task. The power controller 732 receives power from an external source and conditions or modifies the power, as needed, to drive a transducer or to power a light source. The network controller 734 allows the PIM 730 to communicate with outside components, such as image processing 730 (discussed below), The network controller 734 may be configured to operate wirelessly (e.g., WIFI or 4G), with a wired connection, or through an optical connection, which will allow MHz signals to be processed easier away from the PIM 730. The imaging controller 736 will coordinate production of acoustic energy and reception of the reflected energy, as needed to image the tissues. The diagnostic controller 738 will coordinate measurement of diagnostic values, such as blood flow, blood pressure, temperature, or blood oxygenation, for example by interacting with Doppler sensor 160, The therapy controller 740 will control therapy delivery, for example acoustic or photo therapy, delivered with the distal end of the catheter 710.

At least a portion of the output from the PIM 730 will be directed to image processing 760 prior to being output to a display 770 for viewing. The image processing will deconvolve received signals to produce distance and/or tissue measurements, and those distance and tissue measurements will be used to produce an image, for example an intravascular ultrasound (IVUS) image. The image processing may additionally include spectral analysis, i.e., examining the energy of the returned acoustic signal at various frequencies. Spectral analysis is useful for determining the nature of the tissue and the presence of foreign objects. A plaque deposit, for example, will typically have a different spectral signature than nearby vascular tissue without such plaque, allowing discrimination between healthy and diseased tissue. Also a metal surface, such as a stent, will have a different spectral signal. Such signal processing may additionally include statistical processing (e.g., averaging, filtering, or the like) of the returned ultrasound signal in the time domain. Other signal processing techniques known in the art of tissue characterization may also be applied.

Other image processing may facilitate use of the images or identification of features of interest. For example, the border of a lumen may be highlighted or plaque deposits may be displayed in a visually different manner (e.g., by assigning plaque deposits a discernible color) than other portions of the image. Other image enhancement techniques known in the art of imaging may also be applied. In a further example, similar techniques can be used to discriminate between vulnerable plaque and other plaque, or to enhance the displayed image by providing visual indicators to assist the user in discriminating between vulnerable and other plaque. Other measurements, such as flow rates or pressure may be displayed using color mapping or by displaying numerical values.

In other embodiments, a system may comprise a vacuum aspiration pump or additional mechanical components, e.g., rotary power, as needed to achieve the desired procedures.

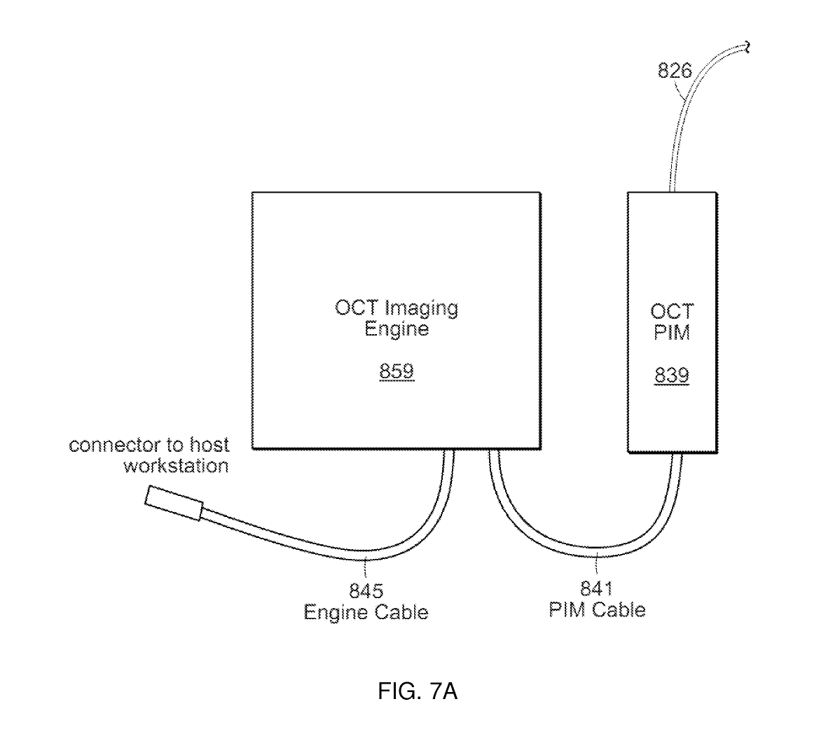

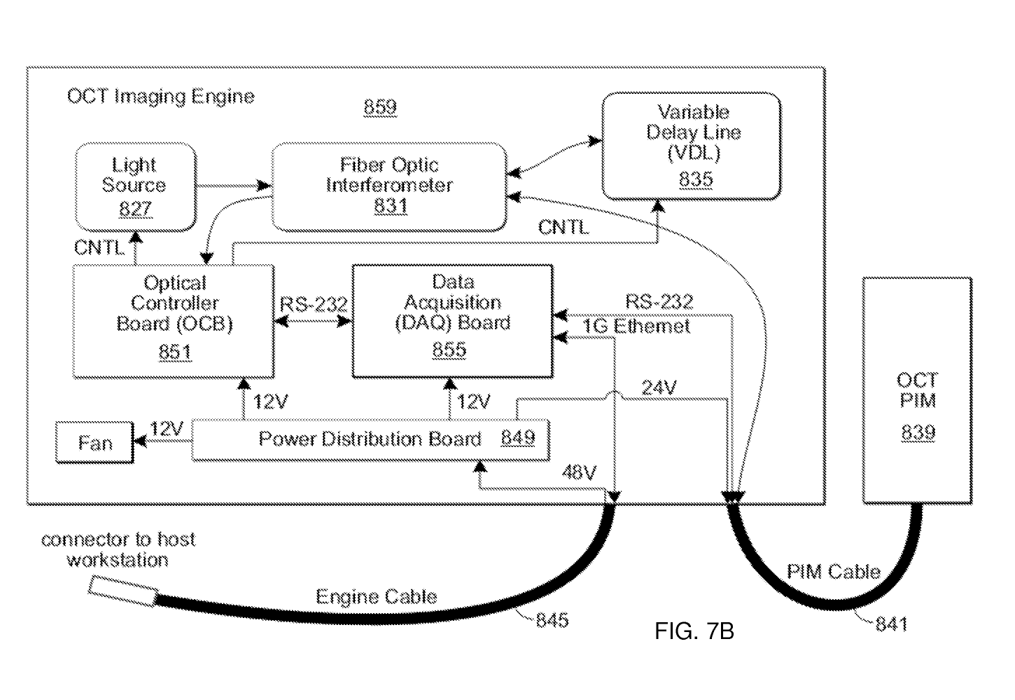

In embodiments using OCT, the system 700 will additionally comprise an OCT subsystem, depicted in FIGS. 7A and 7B. Generally, an OCT system comprises three components which are 1) an imaging catheter 2) OCT imaging hardware, 3) host application software. When utilized, the components are capable of obtaining OCT data, processing OCT data, and transmitting captured data to a host system. OCT systems and methods are generally described in Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety. In certain embodiments, systems and methods of the invention include processing hardware configured to interact with more than one different three dimensional imaging system so that the tissue imaging devices and methods described here in can be alternatively used with OCT, IVUS, or other hardware.

In OCT, a light source delivers a beam of light to an imaging device to image target tissue. Light sources can be broad spectrum light sources, or provide a more limited spectrum of wavelengths, e.g., near infra-red. The light sources may be pulsed or continuous wave. For example the light source may be a diode (e.g., superluminescent diode), or a diode array, a semiconductor laser, an ultrashort pulsed laser, or supercontinuum light source. Typically the light source is filtered and allows a user to select a wavelength of light to be amplified. Wavelengths commonly used in medical applications include near-infrared light, for example between about 800 nm and about 1700 nm. Methods of the invention apply to image data obtained from obtained from any OCT system, including OCT systems that operate in either the time domain or frequency (high definition) domain.

In time-domain OCT, an interference spectrum is obtained by moving a scanning optic, such as a reference mirror, longitudinally to change the reference path and match multiple optical paths due to reflections of the light within the sample. The signal giving the reflectivity is sampled over time, and light traveling at a specific distance creates interference in the detector. Moving the scanning mechanism laterally (or rotationally) across the sample produces reflectance distributions of the sample (i.e., an imaging data set) from which two-dimensional and three-dimensional images can be produced.

In frequency domain OCT, a light source capable of emitting a range of optical frequencies passes through an interferometer, where the interferometer combines the light returned from a sample with a reference beam of light from the same source, and the intensity of the combined light is recorded as a function of optical frequency to form an interference spectrum. A Fourier transform of the interference spectrum provides the reflectance distribution along the depth within the sample.

Several methods of frequency domain OCT are described in the literature. In spectral-domain OCT (SD-OCT), also sometimes called “Spectral Radar” (Optics Letters, vol. 21, No. 14 (1996) 1087-1089), a grating or prism or other means is used to disperse the output of the interferometer into its optical frequency components. The intensities of these separated components are measured using an array of optical detectors, each detector receiving an optical frequency or a fractional range of optical frequencies. The set of measurements from these optical detectors forms an interference spectrum (Smith, L. M. and C. C. Dobson, Applied Optics vol. 28: (1989) 3339-3342), wherein the distance to a scatterer is determined by the wavelength dependent fringe spacing within the power spectrum. SD-OCT has enabled the determination of distance and scattering intensity of multiple scatters lying along the illumination axis by analyzing the exposure of an array of optical detectors so that no scanning in depth is necessary.

Alternatively, in swept-source OCT, the interference spectrum is recorded by using a source with adjustable optical frequency, with the optical frequency of the source swept through a range of optical frequencies, and recording the interfered light intensity as a function of time during the sweep. An example of swept-source OCT is described in U.S. Pat. No. 5,321,501.

Time- and frequency-domain systems can further vary based upon the optical layout of the systems: common beam path systems and differential beam path systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are described in U.S. Pat. No. 7,999,938; U.S. Pat. No. 7,995,210; and U.S. Pat. No. 7,787,127 and differential beam path systems are described in U.S. Pat. No. 7,783,337; U.S. Pat. No. 6,134,003; and U.S. Pat. No. 6,421,164, the contents of each of which are incorporated by reference herein in their entireties.

In certain embodiments, the invention provides a differential beam path OCT system with intravascular imaging capability as illustrated in FIG. 7A. For intravascular imaging, a light beam is delivered to the vessel lumen via a fiber-optic based imaging catheter 826, which is a multifunction catheter of the invention. The imaging catheter is connected through hardware to software on a host workstation. The hardware includes imagining engine 859 and a handheld patient interface module (PIM) 839 that includes user controls. The proximal end of imaging catheter 826 is connected to PIM 839, which is connected to imaging engine 859 as shown in FIG. 7A.

An embodiment of imaging engine 859 is shown in FIG. 7B. Imaging engine 859 (i.e., the bedside unit) houses power distribution board 849, light source 827, interferometer 831, and variable delay line 835 as well as a data acquisition (DAQ) board 855 and optical controller board (OCB) 851. PIM cable 841 connects imagining engine 859 to PIM 839 and engine cable 845 connects imaging engine 859 to the host workstation (not shown).

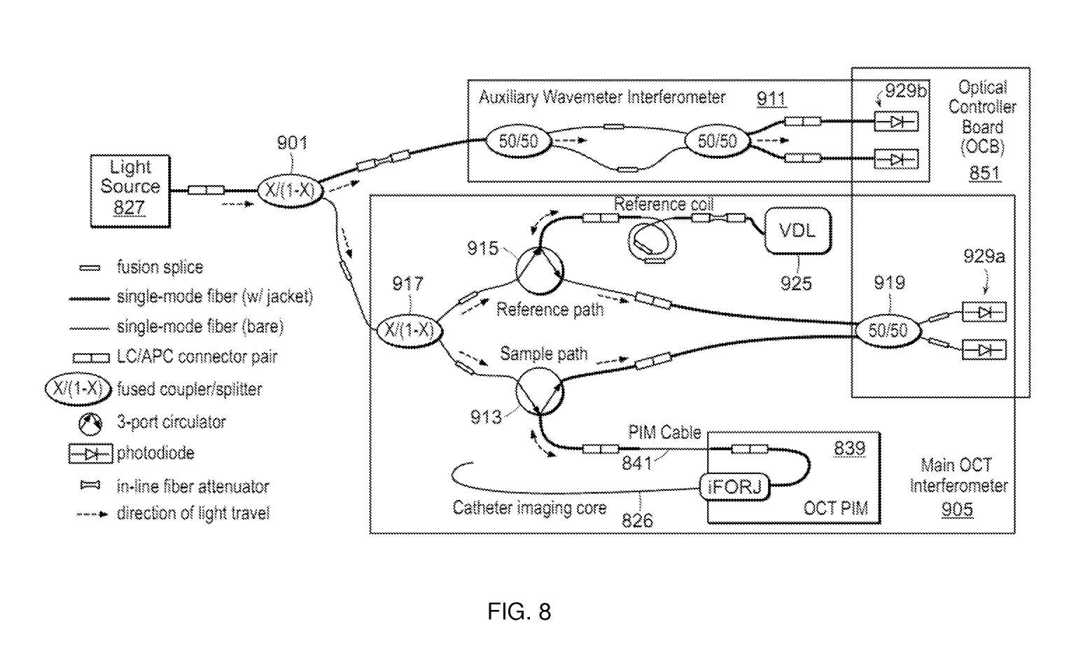

FIG. 8 shows an exemplary light path in a differential beam path system which may be used in an OCT system suitable for use with the invention. Light for producing the measurements originates within light source 827. This light is split between main OCT interferometer 905 and auxiliary interferometer 911. In some embodiments, the auxiliary interferometer is referred to as a “clock” interferometer. Light directed to main OCT interferometer 905 is further split by splitter 917 and recombined by splitter 919 with an asymmetric split ratio. The majority of the light from splitter 917 is guided into sample path 913 while the remainder goes into reference path 915. Sample path 917 includes optical fibers running through PIM 839 and imaging catheter core 826 and terminating at the distal end of the imaging catheter, where the sample is measured.

The reflected light is transmitted along sample path 913 to be recombined with the light from reference path 915 at splitter 919. A variable delay line (VDL) 925 on the reference path uses an adjustable fiber coil to match the length of reference path 915 to the length of sample path 913. The reference path length is adjusted by a stepper motor translating a mirror on a translation stage under the control of firmware or software.

The combined light from splitter 919 is split into orthogonal polarization states, resulting in RF-band polarization-diverse temporal interference fringe signals. The interference fringe signals are converted to photocurrents using PIN photodiodes 929a, and 929b, on optical controller board (OCB) 851. The interfering, polarization splitting, and detection steps are done by a polarization diversity module (PDM) (not shown) on OCB 851. Signal from OCB 851 is sent to DAQ 855, shown in FIG. 6. DAQ 855 includes a digital signal processing (DSP) microprocessor and a field programmable gate array (FPGA) to digitize signals and communicate with the host workstation and PIM 839. The FPGA converts raw optical interference signals into meaningful reflectivity measurements. DAQ 855 also compresses data as necessary to reduce image transfer bandwidth, e.g., to 1 Gbps, e.g., by compressing frames with a glossy compression JPEG encoder.

Additional embodiments of the invention including other combinations of imaging, treatment and assessment will be evident to those of skill in the art in view of this disclosure and the claims below.

### INCORPORATION BY REFERENCE

References and citations to other documents, such as patents, patent applications, patent publications, journals, books, papers, and web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A catheter adapted to image a tissue and deliver a fastener to the tissue.

2. The catheter of claim 1, wherein the catheter is adapted to image the tissue with intravascular ultrasound (IVUS).

3. The catheter of claim 2, wherein the catheter comprises a piezoelectric transducer for IVUS imaging.

4. The catheter of claim 3, wherein the piezoelectric transducer is adapted to deliver focused ultrasonic energy to the tissue being imaged.

5. The catheter of claim 3, wherein the piezoelectric transducer is a piezoelectric micromachined ultrasonic transducer (PMUT).

6. The catheter of claim 3, wherein the piezoelectric transducer is a capacitive micromachined ultrasonic transducer (CMUT).

7. The catheter of claim 1, wherein the catheter is adapted to image the tissue with optical coherence tomography (OCT).

8. The catheter of claim 1, wherein the catheter is adapted to image the tissue with visible imaging.

9. The catheter of claim 1, wherein the fastener is a helical fastener, a screw, a clasp, a tie, or a staple.

10. The catheter of claim 1, wherein the fastener is adapted to secure a strut, stent, valve, graft, electrode, or filter.

11. The catheter of claim 1, wherein the catheter is configured to deliver the fastener with a rotary element at a distal end of the catheter.

12. The catheter of claim 1, wherein the catheter is additionally configured to make a spectroscopic measurement selected from infrared absorption, visible absorption, Raman, or fluorescence.

13. The catheter of claim 1, additionally comprising a radiopaque label.

14. A method of securing a tissue, comprising
imaging a tissue with energy from a catheter;
delivering a fastener to the tissue with the catheter; and
re-imaging the tissue with energy from the catheter.

15. The method of claim 14, wherein the imaging comprises IVUS.

16. The method of claim 14, wherein the imaging comprises focused acoustic computed tomography (FACT).

17. The method of claim 14, wherein the imaging comprises OCT.

18. The method of claim 14, wherein the fastener is a helical fastener, a screw, a clasp, a tie, or a staple.

19. The method of claim 14, further comprising delivering a strut, stent, valve, graft, electrode, or filter to the tissue.

20. The method of claim 14, further comprising measuring a property of an anatomical environment in proximity to the tissue with the catheter.

21. The method of claim 20, wherein the property is blood flow in a vessel, blood pressure in a vessel, blood oxygenation in a vessel, temperature, or a combination thereof.
